(19) United States
(12) Patent Application Publication
(10) Pub. No.: US 2025/0073070 A1
(43) Pub. Date: Mar. 6, 2025

(54) AN OCULAR IMPLANT FOR THE TREATMENT OF GLAUCOMA

(71) Applicants: Yu Xiang George KONG, Mount Waverley (AU); Graham LEE, Sydney, New South Wales (AU)

(72) Inventors: Yu Xiang George KONG, Mount Waverley (AU); Yuanbo LIANG, Mount Waverley (AU); Graham LEE, Mount Waverley (AU)

(21) Appl. No.: 18/713,448

(22) PCT Filed: Dec. 2, 2022
(86) PCT No.: PCT/AU2022/051448

§ 371 Date: May 24, 2024

(30) Foreign Application Priority Data
Dec. 7, 2021 (AU) 2021903948

(51) Int. Cl. A61F 9/007

(52) U.S. Cl. CPC A61F 9/00781; A61F 2230/0008

(57) ABSTRACT

An ocular implant for draining aqueous humour from an anterior chamber of the eye for the treatment of glaucoma has a plate and a drainage tube extending from the plate. The drainage tube has a distal end forming an inlet and a proximal end forming an outlet, the outlet fluidly interfacing the plate. The drainage tube defines a side drainage outlet between the inlet and the outlet and wherein the side drainage outlet is positioned closer to the outlet than the inlet so that when the plate is implanted within subconjunctival space, the side drainage outlet locates within the subconjunctival space so that aqueous humour can drain both from the outlet and the side drainage outlet within the subconjunctival space.

### FIELD OF THE INVENTION

This invention relates generally to ocular implants for the treatment of glaucoma.

### BACKGROUND OF THE INVENTION

Aqueous humour is a fluid formed by the ciliary body in the eye and fills anterior and posterior chambers of the eye.

Aqueous humour produced by the ciliary processes circulates from the posterior chamber to the anterior chamber of the eye through the pupil and is absorbed through the trabecular meshwork whereafter it is drained through Schlemm's canal and into venous circulation.

Glaucoma is a progressive disease of the eye characterised by a gradual increase of intraocular pressure either by stenosis or blockage of the aqueous outflow channel of increase in venous pressure outside the eye which is transferred into the eye through the aqueous drainage channels.

Ocular implants are used for long-term glaucoma treatment without disadvantages of drugs and invasive surgery. One such implant is disclosed in U.S. Pat. No. 4,457,757 entitled “Device for Draining Aqueous Humor” and commercially available as the Molteno™ Seton Implant.

These types of implants comprise a drainage tube connected to a ridged plate reservoir which is designed to conform to the curvature of the eye and which is placed under the Tenon's capsule and sutured to the sclera. A distal end of the drainage tube is tunnelled through the limbus into the anterior chamber.

As aqueous humour drains from the anterior chamber to the rigid plate, increased pressure causes the tissues above the plate to lift and form a bleb. The aqueous humour seeps from the bleb into intercellular spaces and is removed by surrounding capillaries or lymhpatics.

The present invention seeks to provide a way which will overcome or substantially ameliorate at least some of the deficiencies of the prior art, or to at least provide an alternative.

### SUMMARY OF THE DISCLOSURE

There is provided herein an implant for draining aqueous humour from an anterior chamber of the eye for the treatment of glaucoma.

The implant comprises a plate and a drainage tube extending from the plate.

The drainage tube has a distal end forming an inlet and a proximal end forming an outlet. The outlet fluidly interfaces the plate.

Subconjunctival scarring caused by irritation and/or damage to adjacent vasculature and tissue may sometimes encase the rigid plate, thereby hindering drainage and causing pressure buildup in the anterior chamber, requiring revision surgery.

As such, the drainage tube defines a side drainage outlet between the inlet and the outlet. The side drainage outlet is positioned closer to the outlet than the inlet so that when the implant is implanted, the side drainage outlet locates within the subconjunctival space so that aqueous humour can drain both from the outlet across the plate and from the side drainage outlet within the subconjunctival space.

As such, the side drainage outlet provides a backup drainage pathway to mitigate against occlusion of the outlet caused by encapsulation of the plate, thereby reducing the likelihood of revision surgery.

The drainage tube may comprise more than one side drainage outlet spaced along the length of the drainage tube to further mitigate against encapsulation along the tube. For example, collocating drainage outlets may have an increased risk of localised encapsulation caused by irritation of the drainage tube as compared to spacing of the drainage outlets along the length of the tube.

In embodiments, the implant further comprises a side channel for the side drainage outlet which extends out from a surface of the tube to further reduce likelihood of blockage and/or encapsulation. The channel may be tubular as is shown in FIG. 25 or may take the form of a semicircular protrusion which partially surrounds the outlet as is shown FIG. 24.

Furthermore, whereas prior art rigid plate implants are disadvantageous in requiring relatively large incisions to be made and associated surgical trauma of the conjunctival surface, in embodiments, the present implant is configured to be minimally invasive wherein the plate defines a central portion substantially aligned with a longitudinal axis of the proximal end of the drainage tube and side wings either side of the central portion and wherein the plate is elastomeric so that the side wings can be folded over the central portion. The plate may comprise fold lines (or other lines of weakness) generally aligned with a longitudinal axis of the tube, which may assist the folding over of the side wings so that the plate can essentially be folded in three to reduce the width by approximately two thirds.

As such, both side wings can be folded over to form the plate into a relatively compact configuration assuming about third of the unfolded width of the plate so that the plate can be inserted in the compact configuration through a much smaller conjunctival opening. In this way an opening of approximately 5 mm may be adequate as opposed to prior art arrangements which may require an opening of approximately 15 mm or more. Furthermore, this configuration allows the plate to open up much wider than the incision within the subconjunctival space including wherein the plate is sufficiently wide so that distal ends of the wings can insert under respective rectus muscles. The plate may be substantially spade shaped with a leading edge thereof substantially rounded which aids insertion through the opening.

In a further embodiment, the present implant is configured for sutureless implantation wherein an undersurface of the plate is textured at the central portion and smooth at the side wings.

The textured undersurface of the central portion frictionally engages the sclera, thereby reducing likelihood of implant migration within the subconjunctival space.

However, the smooth wings reduced likelihood of irritation and scar tissue formation of the sclera.

Furthermore, the smooth wings allow the wings to be smoothly unfolded inside the subconjunctival space. Specifically, once the implant is inserted into the subconjunctival space in the compact configuration, a blunt flat instrument (such as a spatula or the like) can be inserted via the incision and moved side-to-side to unfold the side wings. The smooth undersurface of the side wings allows the side wings to unfold without frictionally engaging surrounding subconjunctival tissue.

The drainage tube is further preferably textured to provide additional purchase against the sclera to allow for sutureless implantation. In a further preferred embodiment, only an undersurface (i.e., orientated towards the sclera) of the drainage tube is textured so that the undersurface frictionally engages the sclera but wherein an upper surface thereof is smooth to reduce the likelihood of tissue irritation and scar tissue formation.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a perspective view of an implant for the treatment of glaucoma in accordance with an embodiment;

FIG. 2 illustrates the implantation of the implant in an eye;

FIG. 3 shows a side view of the implant;

FIG. 4 shows a side view of the implantation of the implant within a subconjunctival space;

FIG. 5 shows a longitudinal cross-sectional view of a drainage tube of the implant

FIG. 6 shows a portion of the drainage tube of the implant comprising side drainage apertures in accordance with an embodiment

FIG. 7 shows a longitudinal cross-sectional view of the drainage tube with exemplary dimensions;

FIGS. 8-18 illustrate implantation of the implant;

FIG. 19 shows an upper surface of the implant (i.e., away from the sclera);

FIG. 20 shows an undersurface of the implant (i.e., towards the sclera);

FIG. 21 shows a perspective view of the implant with lines of weakness in accordance with an embodiment;

FIG. 22 shows a cross-section of the plate of the embodiment of FIG. 21;

FIG. 23 shows a top view of the implant wherein the implant comprises side channels for the side drainage outlets;

FIG. 24 shows a type of side channel in accordance with an embodiment;

FIG. 25 shows a further type of side channel in accordance with an embodiment; and

FIGS. 26 and 27 illustrate the optional placement of sutures in the vicinity of the side drainage outlet.

### DESCRIPTION OF EMBODIMENTS

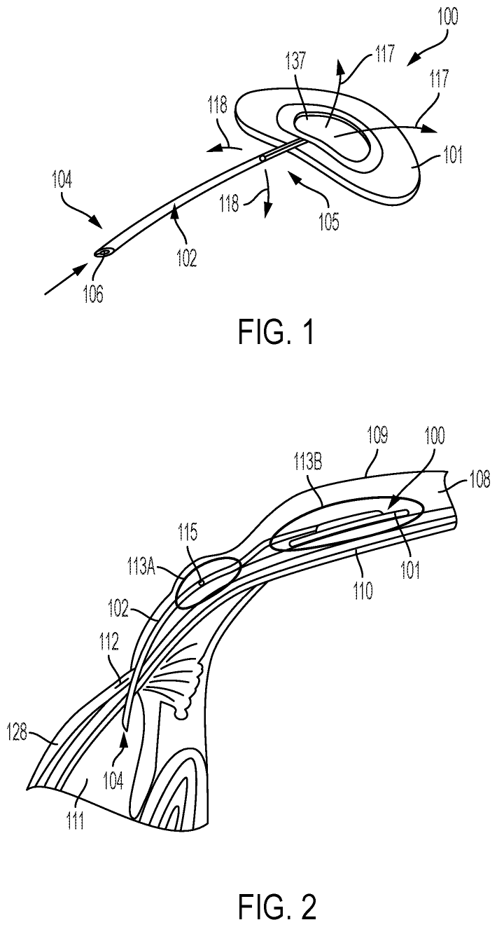

FIG. 1 shows an implant 100 the treatment of glaucoma. The implant 100 comprises a plate 101 and a drainage tube 102 extending from the plate 101. The drainage tube 102 forms a lumen 103 therethrough to transport aqueous humour along the length of the drainage tube 102.

The drainage tube 102 has a distal end 104 forming an inlet 106 and a proximal end 105 forming an outlet 107.

The outlet 107 fluidly interfaces the plate 101 so that aqueous humour can flow from the outlet 107 across the plate 101.

The plate 101 may define a well 137 within which aqueous humour can pool for drainage. With reference to FIG. 4, the well 137 may be formed by a wall 138 raised from an upper surface of the plate 101. The implant 100 may be inserted with the well 137 located away from the sclera 110.

FIG. 2 illustrates the implantation of the implant 100 wherein the plate 101 is inserted into a subconjunctival space 108 between a layer of Tenon's capsule 109 and the sclera 110.

The distal end 104 of the drainage tube 102 is tunnelled through the limbus 112 into the anterior chamber 111.

Aqueous humour within the anterior chamber 111 enters the inlet 106 of the distal end 104 of the drainage tube 102 and dissipates into the subconjunctival space 108, thereby forming a bleb 113.

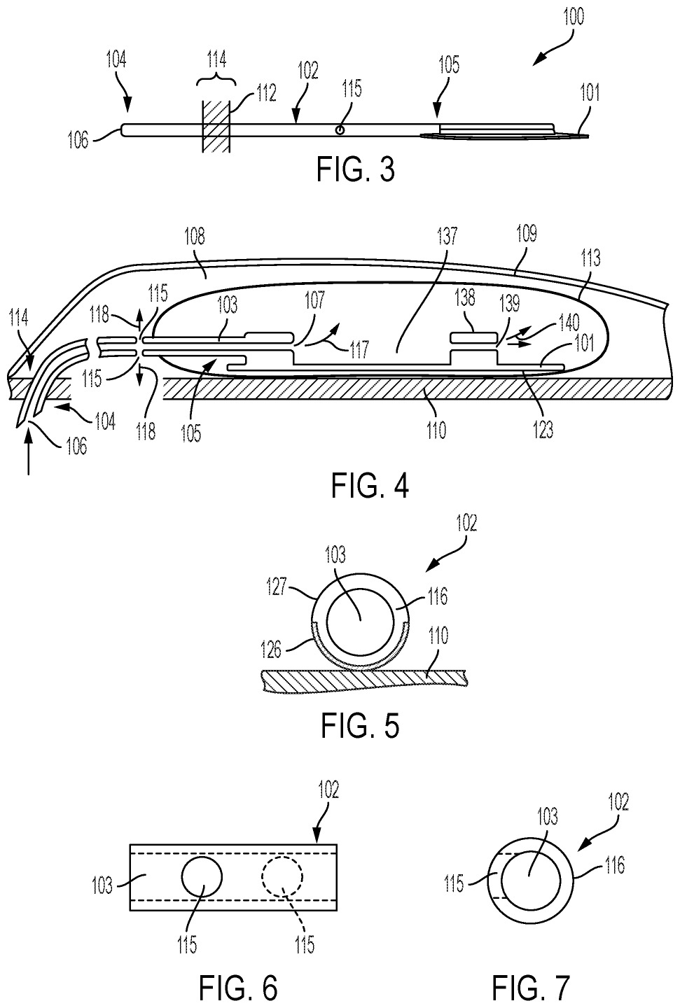

FIG. 3 shows wherein the drainage tube 102 comprises a side drainage outlet 115 through a side wall 116 of the drainage tube 102 between the inlet 106 and the outlet 107.

The side drainage outlet 115 is located along the drainage tube 102 so that the side drainage outlet 115 locates within the subconjunctival space 108. Specifically, with reference to FIG. 3, there is shown a limbus insertion location 114 wherein the drainage tube 102 transitions through the limbus 112.

As can be further seen from FIG. 4, the side drainage outlet 115 is located within the subconjunctival space 108 (i,e, outside of the sclera 110 and beyond the limbus insertion location 114). According to this arrangement, as is shown in FIGS. 1 and 4, the implant 100 provides a primary outflow drainage 117 at the plate 101 and secondary outflow drainage 118 away from the plate 101 within the subconjunctival space 108. The secondary outflow drainage 118 may be away from the bleb 113 and may form a separate bleb 113. In embodiments, the drainage aperture 115 may locate more approximately 5 mm from an edge of the well 137 so that the secondary outflow drainage 118 is sufficiently far from the primary outflow drainage 117 to mitigate against encapsulation.

FIG. 1 illustrates how the primary outflow drainage 117 may form a posterior bleb 113B whereas the secondary outflow drainage 118 from the side drainage outlet 115 may form an anterior bleb 113A. Both blebs 113A and 113B locate within the subconjunctival space 108.

The secondary outflow drainage 118 provides a backup drainage pathway to mitigate against occlusion of the primary outflow drainage 117 caused by subconjunctival scarring of the like.

As is further shown in FIG. 4, the wall 138 may comprise a drainage aperture 139 therethrough to provide further outflow drainage 140. The wall 138 may comprise a plurality of drainage apertures 139 therethrough.

According to FIG. 6, there is shown an embodiment wherein the drainage tube 102 comprises side drainage outlets 115 on opposite sides of the drainage tube 102. Furthermore, the side drainage outlets 115 may be spaced longitudinally along the drainage tube 102 to reduce the likelihood of scarring occluding collocating apertures 115.

As is shown in FIG. 6, each drainage aperture 115 may comprise a diameter of approximately 0.07 mm and may be spaced apart by the same amount.

As is further shown in FIG. 6, the drainage tube 102 may comprise an interior diameter of approximately 0.08 mm and an exterior diameter of approximately 0.130 mm. This interior diameter is selected according to Poiseuille's equation to limit the flow of aqueous humour through the lumen 103 to maintain a minimum residual pressure within the anterior chamber 111.

In the embodiment shown in FIGS. 3, 6 and 7, the side drainage outlet takes the form of a micro hole formed through the side wall 116 of the tube 102.

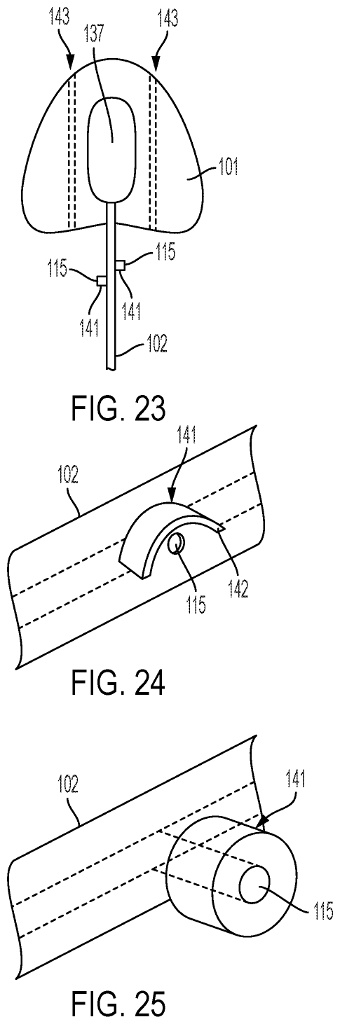

However, FIGS. 23-25 show an embodiment wherein the implant 100 further comprises a side channel 141 for the side drainage outlet 115. The side channel 141 extends out from a surface of the tube. The side channel 141 further protects the side drainage outlet 115 from occlusion.

In the embodiment shown In FIG. 24, the side channel 141 takes the form of a protrusion 142 adjacent the outlet 115. The embodiment shown in FIG. 24 shows wherein the protrusion 124 is semicircular so as to partially surround the outlet 115.

FIG. 25 shows an embodiment wherein the side channel 141 is tubular and wherein the outlet 115 is formed therethrough.

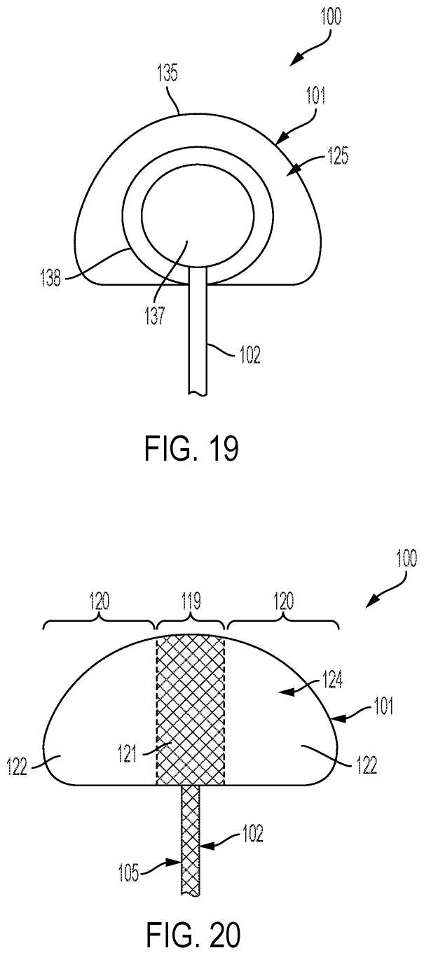

FIG. 19 shows an upper surface 125 of the plate 101 and FIG. 20 shows an undersurface 124 thereof. As shown in FIG. 4, the undersurface 123 may be placed against the sclera 110 within the subconjunctival space 108.

According to embodiments, the plate 101 is flexible. For example, the plate 101 may comprise an elastomer such as silicon.

Furthermore, as is shown in FIG. 20, the plate 101 may define a central portion 119 generally aligned with a longitudinal axis defined by the proximal end 105 of the drainage tube 102 and side wings 120 either side of the central portion 119.

The flexible plate 101 allows the side wings 120 to be folded over each other and over the central portion 119 to make the implant 100 into a much more compact folded configuration as is shown in FIG. 12. In this compact folded configuration, the plate 101 comprises a width of about a third of the plate when unfolded.

As is evident from FIG. 19, the well 137 may extend across the central portion 119 and the side wings 120 and the well 137 itself may be flexible so that the well 137 can fold when the plate 100 is folded up. According to this embodiment, the generally circular wall 138 defining the well 137 may be integrally formed with the plate 101 such as wherein the plate 101 and the wall 137 is integrally moulded from silicon. As such, the well 137 may be wider than the central portion 137.

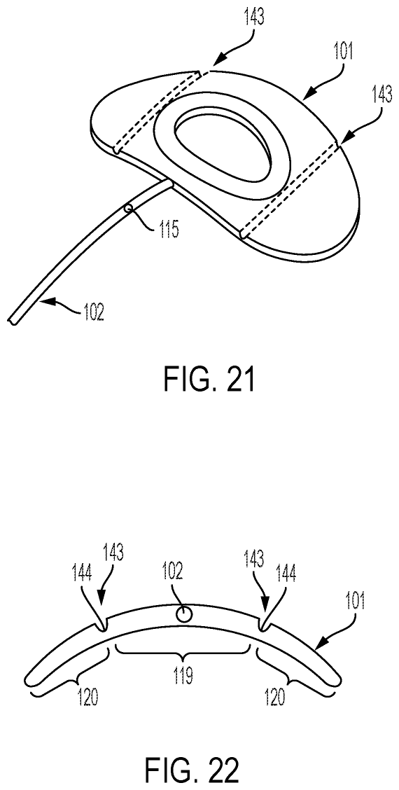

FIGS. 21 and 22 show wherein the plate comprises lines of weakness 143 demarcating the side wings 120 from the central portion 119. As is evident from FIG. 21, the lines of weakness 143 may generally be aligned with a longitudinal axis of the drainage tube 102. As is shown in FIG. 2, the lines of weakness may be formed by score lines/channels 144 formed into a surface of the plate 101.

FIG. 20 shows wherein the undersurface 124 comprises a textured surface 121 at the central portion 119 and smooth surfaces 122 at the side wings 120.

The textured surface 121 at the central portion 119 frictionally engages the sclera 110, thereby reducing potential for migration of the plate 101 and allowing for implanting without use of sutures.

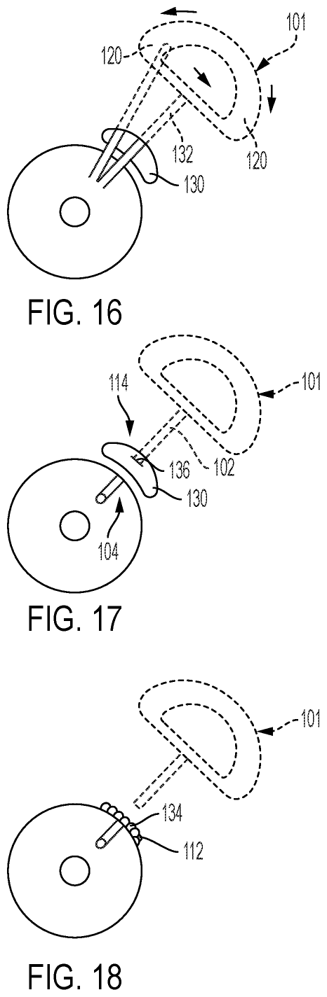

The smooth surfaces 122 of the side wings 120 however reduce the likelihood of scar tissue formation and irritation of the sclera. Furthermore, the smooth surfaces 122 of the side wings 120 allow the side wings 120 to be unfolded in the manner shown in FIG. 16 with a spatula or similar instrument without hindrance as will be described in further detail below.

In a further preferred embodiment, an exterior surface of the drainage tube 102 comprises a textured surface. As such, the textured surface of the drainage tube 102 can work in conjunction with the textured surface of 121 of the central portion 119 to hold the implant 100 in place without sutures.

With reference to FIG. 5, preferably only an undersurface 126 of the drainage tube 102 (that is towards the sclera 110) comprises the textured surface whereas an upper surface 127 thereof is smooth. The textured lower undersurface 126 obtains purchase on the sclera 110 whereas the smooth upper surface 127 reduces the likelihood of irritation and scar tissue formation.

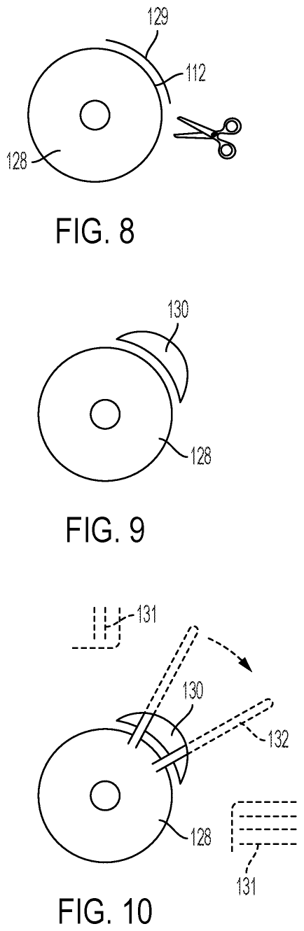

FIGS. 8 to 18 illustrate the implantation of the implant 100 for the treatment of glaucoma.

FIG. 8 shows wherein a relatively small incision 129 is made through the conjunctiva near the limbus 112 and FIG. 9 shows wherein the incision 129 is opened to form an opening 130 into the subconjunctival space 108.

FIG. 10 shows wherein a spatula 132 or other type of generally flat and blunt instrument is inserted through the opening 120 and moved side to side between the rectus muscles 131 to clear a pocket within the subconjunctival space 108.

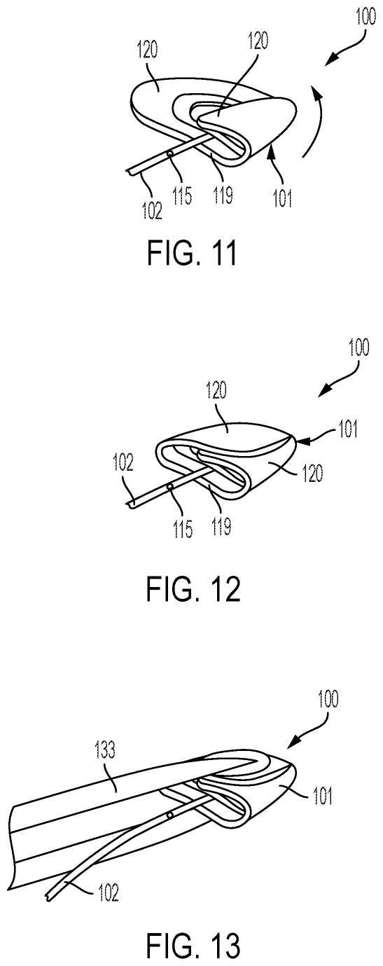

FIG. 11 shows wherein a first side wing 120 of the plate 101 is folded over the central portion 119 and FIG. 12 shows wherein the other side wing 120 is further folded over so that the implant 100 takes the compact configuration shown in FIG. 12. The aforedescribed lines of weakness 143 may assist folding the plate 101 three-ways so that the plate 101 has a folded width of approximately one third of the unfolded width.

With reference to FIG. 19, the plate 101 may be substantially spade shaped having a rounded leading edge 135 which aids insertion through the opening 130.

FIG. 13 shows wherein the folded plate 101 is compactly held between a pair of forceps 133 or the like and wherein the drainage tube 102 runs back away from the tips of the forceps 133.

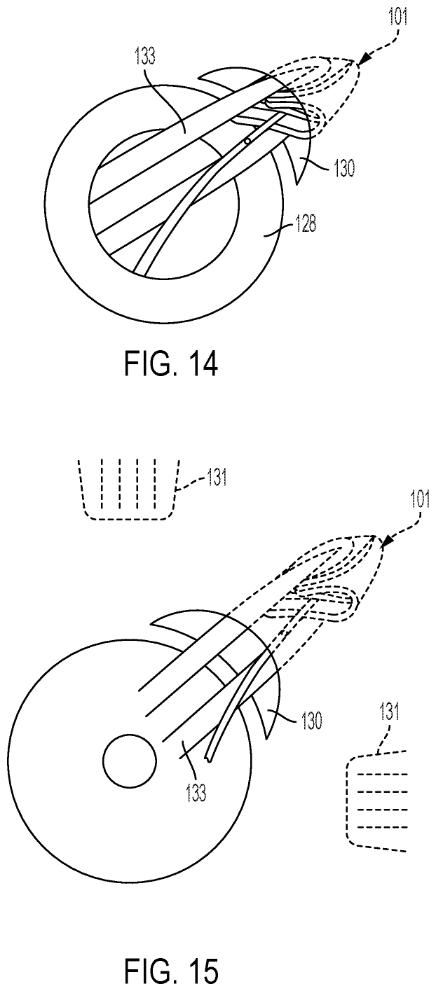

FIG. 14 shows wherein the forceps 133 are used to insert the compactly folded over plate 101 into the opening 130 to a suitable target region within the subconjunctival space 108 as shown in FIG. 15 (and/or to control the length of the tube 102 extending from the opening 130).

The implant 101 is be placed to ensure that the drainage apertures 115 locate within the subconjunctival space 108.

FIG. 16 shows wherein the spatula 132 (or other generally flat blunt instrument) is inserted over the plate 101 and moved side to side to unfold the side wings 120.

Once unfolded as is shown in FIG. 17, the distal end 104 of the drainage tube 102 is inserted through an incision 136 at the limbus insertion location 114 so that the distal end 104 of the drainage tube 102 penetrates the anterior chamber 111.

FIG. 18 shows wherein the opening 130 is closed with stitches 134.

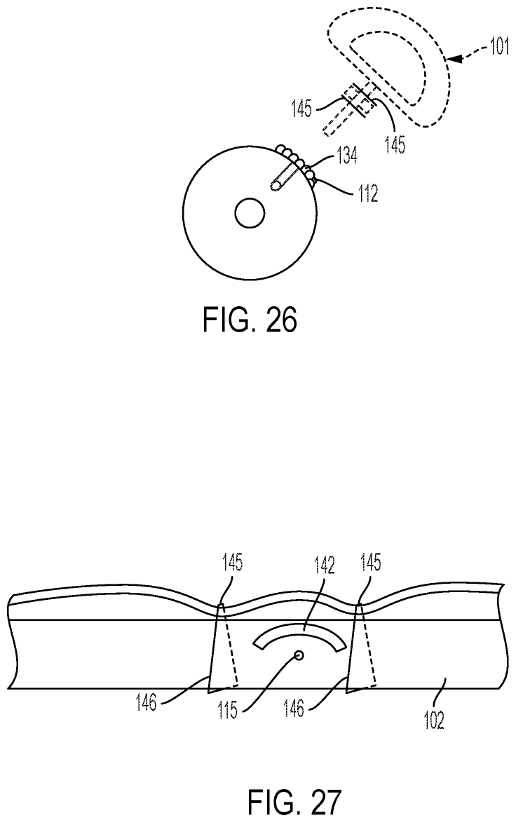

With reference to FIGS. 26 and 27, sutures 145 may optionally be placed trans-conjunctivally at this stage. Specifically, as is shown in FIG. 26, one or two sutures 145 may be are passed through the conjunctiva and partial thickness of sclera to form suture loops 146 around the drainage tube 102 in the vicinity of the side drainage outlet 115. These sutures 145 and loops provide extra security to prevent movement of the implant 100 in the post-operative period.

In embodiments shown in FIG. 27, a pair of loops 146 of formed by the sutures 145 either side of the side drainage outlet 115.

As can be appreciated from the foregoing, the relatively small incision 129 may be made through the conjunctiva, such as an incision of about 5 mm in length, as opposed to the conventional length of approximately 15-20 mm or more.

Furthermore, the way in which the side wings 101 can fold over the central portion 119 (wherein such folding may be aided by the lines of weakness 143) allows the plate 101 to assume a folded width of approximately a third of an unfolded width to fit through the smaller opening 130 and then expand substantially within the subconjunctival space 108. Preferably, the plate 101 is wide enough so that ends of the wings 121 can fit under the respective rectus muscles 131.

Once implanted, aqueous humour within the anterior chamber 111 will flow through the inlet 106 of the distal end 104 of the drainage tube 102, travel through the lumen 103 thereof and dissipate within the subconjunctival space 108.

Aqueous humour may escape from the outlet 107 across the plate 101 within the posterior bleb 113B. Further aqueous humour may escape via the side drainage outlets 115 away from the plate 101 and/or the posterior bleb 113B, and which may form its own interior bleb 113A, thereby providing backup in case of occlusion of the outlet 107 or scar tissue formation around the plate 101. Further aqueous humour may escape via the drainage apertures 139 of the wall 138.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. An ocular implant for draining aqueous humour from an anterior chamber of the eye for the treatment of glaucoma, the implant comprising a plate and a drainage tube extending from the plate, the drainage tube having a distal end forming an inlet and a proximal end forming an outlet fluidly interfacing the plate and wherein the drainage tube defines a side drainage outlet adjacent the plate so that when the plate is implanted within subconjunctival space, the side drainage outlet locates within the subconjunctival space so that aqueous humour can drain both from the outlet and the side drainage outlet within the subconjunctival space.

2. The implant as claimed in claim 1, wherein the side drainage outlet is located approximately a quarter of a length of the drainage tube away from the outlet.

3. The implant as claimed in claim 1, wherein the side drainage outlet is located less than 8 mm from the outlet.

4. The implant as claimed in claim 1, wherein the side drainage outlet is located approximately 5 mm from the outlet.

5. The implant as claimed in claim 1, wherein the drainage tube defines two side drainage outlets which are offset along a length of the drainage tube.

6. The implant as claimed in claim 1, further comprising a side channel for the side drainage outlet, wherein the channel protrudes out from a surface of the tube.

7. The implant as claimed in claim 6, wherein the channel is tubular and wherein the side drainage outlet is formed therethrough.

8. The implant as claimed in claim 6, wherein the channel is formed by a protrusion adjacent the side drainage outlet.

9. The implant as claimed in claim 8, wherein the protrusion is semicircular.

10. The implant as claimed in claim 1, wherein the plate defines a central portion substantially aligned with a longitudinal axis of the proximal end of the tube and side wings either side of the central portion and wherein the plate is elastomeric so that the side wings can be folded over the central portion.

11. The implant as claimed in claim 10, further comprising lines of weakness between the central portion and the side wings.

12. The implant as claimed in claim 11, wherein the lines of weakness are generally orientated parallel with a longitudinal axis of the drainage tube.

13. The implant as claimed in claim 11, wherein the lines of weakness are formed by channels indented within a surface of the plate.

14. The implant as claimed in claim 10, wherein an undersurface of the plate is textured at the central portion and smooth at the side wings.

15. The implant as claimed in claim 1, wherein at least a portion of an exterior surface of the drainage tube is textured.

16. The implant as claimed in claim 8, wherein an undersurface of the drainage tube is textured and an upper surface of the drainage tube is smooth.

17. The implant as claimed in claim 10, wherein the plate defines a well and wherein the well extends across the central portion and the side wings.

18. The implant as claimed in claim 17, wherein the well is formed by a wall raised from an upper surface of the plate and wherein the wall and the plate are integrally formed.

19. The implant as claimed in claim 1, wherein the plate defines a well formed by a wall raised from an upper surface of the plate and wherein the wall comprises a drainage perforation between an inner side and an outer side thereof.

20. The implant as claimed in claim 1, wherein trans-conjunctival sutures are passed through the conjunctiva and partial thickness of sclera to form suture loops around the drainage tube in the vicinity of the side drainage outlet.

21. The implant as claimed in claim 20, wherein a pair of loops are formed either side of the side drainage outlet.

22. A method of implanting the implant as claimed in claim 1, the method comprising implanting the implant so that the plate and the side drainage outlet-locate within the subconjunctival space to thereby both provide primary and secondary drainage respectively within the subconjunctival space.

23. A method as claimed in claim 22, wherein the plate defines a well formed by a wall raised from an upper surface of the plate and wherein the wall comprises a drainage perforation between an inner side and an outer side thereof and wherein the drainage perforation provides further drainage.

24. A method as claimed in claim 22, wherein the plate defines a central portion substantially aligned with a longitudinal axis of the proximal end of the tube and side wings either side of the central portion and wherein the plate is elastomeric so that the side wings can be folded over the central portion and wherein the method further comprises folding the side wings of the central portion to make the plate into a compact configuration, inserting the plate when in the compact configuration through a conjunctival opening wherein the side wings are unfolded within the subconjunctival space.

25. The method as claimed in claim 24, wherein the method further comprises inserting a flat instrument through the opening and moving the flat instrument side to side to unfold the side wings.

26. The method as claimed in claim 24, wherein the implant further comprising lines of weakness between the central portion and the side wings and wherein the method further comprises folding the plate along the lines of weakness.

27. A method of implanting the implant as claimed in claim 10, the method comprising folding the side wings of the central portion to make the plate into a compact configuration, inserting the plate when in the compact configuration through a conjunctival opening wherein the side wings are unfolded within the subconjunctival space and wherein the plate is not secured to the sclera with stitches.
